(19) United States
(12) Patent Application Publication
(10) Pub. No.: US 2023/0050667 A1
(43) Pub. Date: Feb. 16, 2023

(54) Concentration Device

(71) Applicant: Nippon Telegraph and Telephone Corporation, Tokyo (JP)

(72) Inventors: Yuki Hashimoto, Tokyo (JP); Takako Ishihara, Tokyo (JP); Kei Kuwabara, Tokyo (JP)

(21) Appl. No.: 17/792,984

(22) PCT Filed: Jan. 29, 2020
(86) PCT No.: PCT/JP2020/003147

§ 371 Date: Jul. 14, 2022

(51) Int. Cl. G01N 1/40; B01D 1/30; B01D 53/04

(52) U.S. Cl. CPC G01N 1/405; B01D 1/30; B01D 53/0407; B01D 2253/102; B01D 2253/106; B01D 2253/108; G01N 2001/4066

(57) ABSTRACT

A concentration device includes a substrate that has a flow path formed therein extending from an inlet-side opening into which a liquid to be concentrated is introduced to an outlet-side opening, and a desiccant that is disposed in containing spaces in the substrate to face a liquid in the flow path across air layers.

### CROSS-REFERENCE TO RELATED APPLICATIONS

This application is a national phase entry of PCT Application No. PCT/JP2020/003147, filed on Jan. 29, 2020, which application is hereby incorporated herein by reference.

### TECHNICAL FIELD

The present invention relates to a concentration device that concentrates a liquid.

### BACKGROUND

Sweating sensors have recently attracted attention because they can detect components such as electrolyte ions, alcohols, glucose, urea, lactic acid, proteins, and hormones contained in body fluids without invasive procedures such as blood tests. Non-Patent Literature 1 discloses a wearable sensor capable of monitoring components of sweat.

Of such components of sweat, sodium and chloride ions are reabsorbed in sweat glands in the process of sweating, and their concentrations in sweat are thus lower than those in blood. Other components of sweat than the sodium and chloride ions are in trace amounts, and need a highly sensitive sensor to be detected (see Non-Patent Literature 2).

### CITATION LIST

### Non-Patent Literature

Non-Patent Literature 1: W. Gao, et al., “Fully integrated wearable sensor arrays for multiplexed in situ perspiration analysis”, nature, Vol. 509, pp. 509-526, 2016; and

Non-Patent Literature 2: Z. Sonner, et al., “The microfluidics of the eccrine sweat gland, including biomarker partitioning, transport, and biosensing implications”, Biomicrofluidics, Vol. 9, 031301, 2015.

### SUMMARY

### Technical Problem

Embodiments of the present invention have been made to solve the above-mentioned problem, and it is an object of embodiments of the present invention to provide a concentration device capable of concentrating a liquid.

### Means for Solving the Problem

A concentration device of embodiments of the present invention includes a substrate that has a first flow path formed therein extending from an inlet-side opening into which a liquid to be concentrated is introduced to an outlet-side opening, and a desiccant that is disposed in containing spaces in the substrate to face a liquid in the first flow path across air layers.

### Effects of embodiments of the Invention

According to embodiments of the present invention, the desiccant adsorbs a solvent vapor generated through evaporation of a liquid containing a non-volatile solute, and the liquid can thereby be concentrated. For example, the use of the concentration device of embodiments of the present invention for detecting components of sweat allows detection sensitivity to the components contained in sweat to be improved, and thus analysis of the components can be achieved without a highly sensitive sensor.

### BRIEF DESCRIPTION OF THE DRAWINGS

FIG. 1 is an outline view of a concentration device according to an embodiment of the present invention.

FIG. 2 is a front view of a flow path member constituting the concentration device according to the embodiment of the present invention.

FIG. 3 is an enlarged view of the flow path member constituting the concentration device according to the embodiment of the present invention.

FIG. 4 is a cross-sectional view of the concentration device according to the embodiment of the present invention.

FIG. 5 illustrates a state in which a liquid to be concentrated is introduced into a flow path through an inlet-side opening of the concentration device and flows in the flow path.

### DETAILED DESCRIPTION OF ILLUSTRATIVE EMBODIMENTS

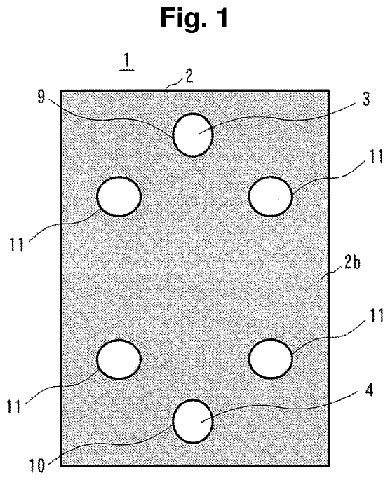

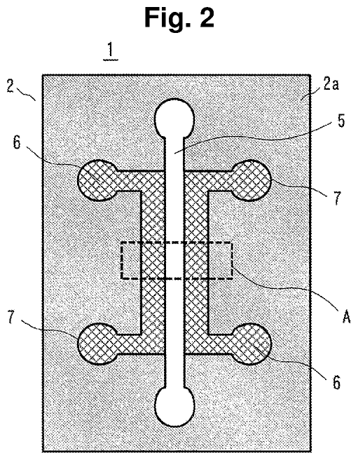

Hereinafter, an embodiment of the present invention will be described with reference to the drawings. FIG. 1 is an outline view of a concentration device according to the embodiment of the present invention, and FIG. 2 is a front view of a flow path member constituting the concentration device. A concentration device 1 of the present embodiment includes a substrate 2 that has a flow path 5 formed therein extending from an inlet-side opening 3 into which a liquid to be concentrated is introduced to an outlet-side opening 4, and a desiccant 6 that is disposed in containing spaces 7 in the substrate 2 to face a liquid in the flow path 5 across air layers.

The substrate 2 consists of a plate-shaped flow path member 2a and a plate-shaped lid member 2b joined to the flow path member 2a.

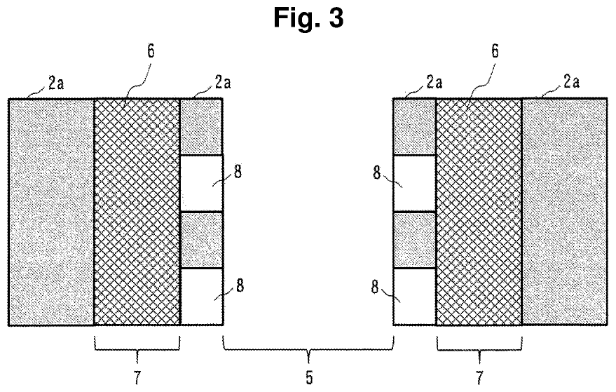

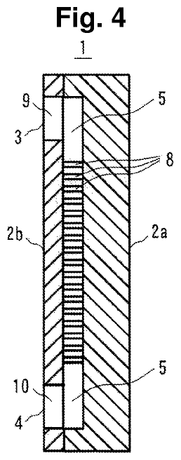

FIG. 3 is an enlarged view of the part A in FIG. 2, and FIG. 4 is a cross-sectional view of the concentration device 1 with the flow path member 2a being joined to the lid member 2b.

In the flow path member 2a, there are formed the flow path 5 in a grooved shape, the containing spaces 7 in a grooved shape that are formed on the opposite sides of the flow path 5 and contain the desiccant 6, and a plurality of flow paths fin a grooved shape that allow communication between the flow path 5 and the containing spaces 7. The flow paths 8 are formed in the side wall of the flow path 5 separating the flow path 5 and the containing spaces 7.

As a result, the desiccant 6 faces the liquid in the flow path 5 across the air layers in the flow paths 8.

In the lid member 2b, a through hole 9 is formed to cut through the lid member 2b from the front surface to the back surface in a position such that the through hole 9 communicates with the inlet-side end of the flow path 5 when the flow path member 2a is joined to the lid member 2b. The opening on the front surface side of the through hole 9 serves as the inlet-side opening 3 of the concentration device 1.

In the lid member 2b, a through hole 10 is also formed to cut through the lid member 2b from the front surface to the back surface in a position such that the through hole 10 communicates with the outlet-side end of the flow path 5 when the flow path member 2a is joined to the lid member 2b. The opening on the front surface side of the through hole 10 serves as the outlet-side opening 4 of the concentration device 1.

In the lid member 2b, vents 11 are further formed to cut through the lid member 2b from the front surface to the back surface in positions such that the vents 11 communicate with the containing spaces 7 when the flow path member 2a is joined to the lid member 2b. The reason for forming the vents 11 is that providing the vents 11 causes the air in the containing spaces 7 to be discharged to decrease the vapor pressure and to facilitate movement of the vapor from the flow path 5 to the desiccant 6 in the containing spaces 7.

Examples of materials available for the flow path member 2a and the lid member 2b include synthetic resins having high water repellence such as polydimethylsiloxanes, cycloolefin polymers, acrylic resins, and polycarbonates. For the flow path member 2a and the lid member 2b, a processed material is also available which has been subjected to a surface treatment for providing water repellence or a coating process for forming a water repellent film onto the surface of any hydrophilic material, the inner surface of the flow path 5, and the inner surfaces of the through holes 9 to 10.

As described above, the desiccant 6 is contained in the grooved containing spaces 7 formed on the opposite sides of the flow path 5 in the flow path member 2a. Examples of the desiccant 6 include water vapor adsorbents such as silica gel, activated alumina, and zeolite.

The flow path member 2a and the lid member 2b are joined to each other so that, with the desiccant 6 contained in the containing spaces 7, the inlet-side end of the flow path 5 communicates with the through hole 9, the outlet-side end of the flow path 5 communicates with the through hole 10, the containing spaces 7 communicate with the vents 11, and the lids of the flow paths 5, 8 are closed. Examples of joining methods include direct joining, adhesive joining, and mechanical joining, but the present invention is not limited to these joining methods.

The flow path member 2a and the lid member 2b may also be integrally formed using a 3D printer, for example.

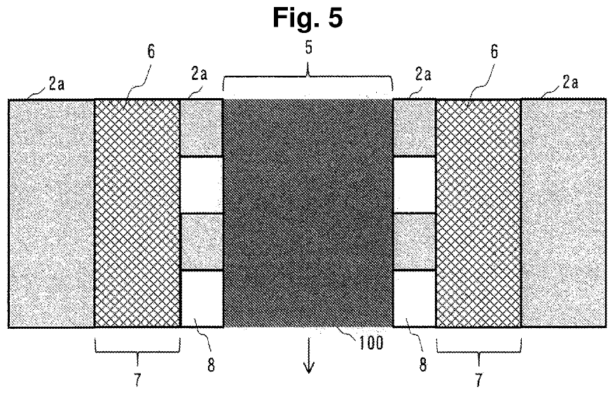

FIG. 5 illustrates a state in which a liquid 100 to be concentrated is introduced into the flow path 5 through the inlet-side opening 3 and flows in the flow path 5.

To facilitate movement of the liquid 100, the concentration device 1 is desirably installed so that the inlet-side opening 3 is located above the outlet-side opening 4 and the flow path 5 is disposed vertically downward or obliquely downward. When the flow path 5 is disposed horizontally, the liquid 100 is desirably fed with a pump.

Because the vapor pressure in the containing spaces 7 is lower than that in the flow path 5, the vapor generated through evaporation of the liquid 100 moves toward the containing spaces 7 through the flow paths 8 and is adsorbed by the desiccant 6 in the containing spaces 7.

In this way, the desiccant 6 adsorbs the solvent vapor generated through evaporation of the liquid 100 containing a non-volatile solute, and the liquid 100 is thereby concentrated and the concentrated liquid 100 can be discharged from the outlet-side opening 4 to the outside.

Although the liquid wo unlikely enters the flow paths 8 because of the water repellence of the flow path member 2a, the opening area and length of the flow paths 8 are desirably determined such that the liquid wo is not allowed to pass through the flow paths 8 and the vapor is allowed to pass through the flow paths 8. The concentration rate of the liquid wo can be adjusted by changing the opening rate of the side wall of the flow path 5 (the ratio of the opening area of the flow paths 8 to the total area of the side wall when the flow paths 8 are not provided in the side wall).

As an exemplary usage of the concentration device 1 of the present embodiment, sweat collected from skin of a subject is introduced into the concentration device 1 and components contained in the concentrated sweat may be detected. A method for detecting concentrations of such components is disclosed in Non-Patent Literature 1. The use of the concentration device 1 of the present embodiment allows detection sensitivity to the components contained in sweat to be improved, and thus analysis of the components can be achieved without a highly sensitive sensor.

The concentration device 1 of the present embodiment is available not only to sweat but also to aqueous solutions in general.

### INDUSTRIAL APPLICABILITY

Embodiments of the present invention are available to techniques for concentrating a liquid.

### REFERENCE SIGNS LIST

2 substrate

2a flow path member

2b lid member

3 inlet-side opening

4 outlet-side opening

5, 8 flow path

6 desiccant

7 containing space

9, 10 through hole

11 vent.

1-5. (canceled)

6. A concentration device comprising:
a substrate that has a first flow path disposed therein extending from an inlet-side opening to an outlet-side opening, wherein the inlet-side opening is configured so that a liquid to be concentrated is introduced into the inlet-side opening; and
a desiccant disposed in containing spaces in the substrate, the desiccant being configured to face a liquid in the first flow path across one or more air layers.

7. The concentration device according to claim 6, wherein the substrate further comprises vents configured to discharge air in the containing spaces.

8. The concentration device according to claim 6, further comprising:
second flow paths extending between the first flow path and the containing spaces, the second flow paths are disposed in a side wall of the first flow path separating the first flow path and the containing spaces, wherein the desiccant faces the liquid in the first flow path across the one or more air layers in the second flow paths.

9. The concentration device according to claim 8, wherein the substrate comprises:
a flow path member in which the first flow path in a grooved shape, the containing spaces in a grooved shape, and the second flow paths in a grooved shape are disposed; and
a lid member in which a first through hole extending from an inlet-side opening of a front surface to a back surface and a second through hole extending from an outlet-side opening of the front surface to the back surface are disposed, and wherein the lid member is joined to the flow path member so that, with the desiccant contained in the containing spaces, an inlet-side end of the first flow path is connected with the first through hole, an outlet-side end of the first flow path is connected with the second through hole, and lids of the first flow path and the second flow paths are closed.

10. The concentration device according to claim 9, wherein the lid member has vents disposed therein configured to discharge air in the containing spaces in a position such that the vents are connected with the containing spaces when the lid member is joined to the flow path member.

11. The concentration device according to claim 6, wherein the desiccant comprises silica gel, activated alumina, or zeolite.

12. A concentration device comprising:
a substrate having a first flow path, the first flow path extending from an inlet-side opening to an outlet-side opening, wherein the inlet-side opening is configured so that a liquid to be concentrated is introduced into the inlet-side opening; and
a desiccant disposed in the substrate, the desiccant being connected to the first flow path by a plurality of second flow paths, each of the plurality of second flow paths being an air channel.

13. The concentration device according to claim 12, wherein the substrate further comprises vents configured to discharge air in containing spaces in which the desiccant is disposed.

14. The concentration device according to claim 12, wherein the substrate comprises:
a flow path member containing the first flow path, the desiccant, and the second flow paths; and
a lid member in which a first through hole extending from an inlet-side opening of a front surface to a back surface and a second through hole extending from an outlet-side opening of the front surface to the back surface are disposed, and wherein the lid member is joined to the flow path member so that, with the desiccant, an inlet-side end of the first flow path is connected with the first through hole, an outlet-side end of the first flow path is connected with the second through hole, and lids of the first flow path and the second flow paths are closed.

15. The concentration device according to claim 14, wherein the lid member has vents disposed therein configured to discharge air in containing spaces of the desiccant in a position such that the vents are connected with the containing spaces when the lid member is joined to the flow path member.

16. The concentration device according to claim 12, wherein the desiccant comprises silica gel, activated alumina, or zeolite.

17. A method comprising:
forming a first flow path in a substrate, the first flow path extending from an inlet-side opening to an outlet-side opening, wherein a liquid to be concentrated is configured to be introduced into the inlet-side opening; and
disposing a desiccant in containing spaces in the substrate, the desiccant being configured to face a liquid in the first flow path across a plurality of air channels.

18. The method of claim 17, further comprising:
forming second flow paths extending between the first flow path and the containing spaces, the second flow paths are disposed in a side wall of the first flow path separating the first flow path and the containing spaces, wherein the desiccant faces the liquid in the first flow path across the plurality of air channels in the second flow paths.

19. The method according to claim 17, wherein the substrate further comprises vents configured to discharge air in the containing spaces.
